(19) United States
(12) Patent Application Publication
(10) Pub. No.: US 2006/0271019 A1
(43) Pub. Date: Nov. 30, 2006

(54) Drainage system

(75) Inventors: Marshall L. Stoller, San Francisco, CA (US); Maurice Garcia, San Francisco, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 11/251,560

(22) Filed: Oct. 14, 2005

Related U.S. Application Data
(60) Provisional application No. 60/619,104, filed on Oct. 15, 2004.

(51) Int. Cl. A61M 27/00

(52) U.S. Cl. 604/544; 604/353

(57) ABSTRACT

This invention provides improved medical drainage devices. In certain embodiments this invention provides a system for improved drainage from a bladder in a patient where the system comprises: a fluid collection apparatus; a drainage receptacle; and a connecting tube comprising a means for reducing or eliminating airlocks in the connecting tube and thereby providing sufficiently low backpressure such that a patient having a urinary bladder drained with said system maintains an average residual bladder urine volume of less than about 50 cubic centimeters over a period of at least four hours after initial drainage without manipulation of components of said system.

### CROSS-REFERENCE TO RELATED APPLICATIONS

This application claims benefit of and priority to U.S. Ser. No. 60/619,304, filed on Oct. 15, 2004 which is incorporated herein by reference in its entirety for all purposes.

### STATEMENT AS TO RIGHTS TO INVENTIONS MADE UNDER FEDERALLY SPONSORED RESEARCH AND DEVELOPMENT

[Not Applicable]

### FIELD OF THE INVENTION

This invention pertains to medical drainage devices.

### BACKGROUND OF THE INVENTION

A urinary drainage catheter, such as the Foley catheter, is a hollow, tubular device commonly used in the medical profession for insertion into a patient's bladder via the urethral tract to permit the drainage of urine. Use of a urinary catheter is often necessary for patients that are undergoing surgery, orthopedically incapacitated, incontinent, or incapable of voluntary urination. An unfortunate problem with catheterization, however, is the development of sepsis and/or urinary tract infections (UTIs) as a result of bacterial invasion in the bladder and urinary tract by various microorganisms. Sepsis is potentially lethal and most prevalent in the elderly, where urinary tract and bladder infections become systemic very easily, especially if hygiene is poor and hydration of tissue is deficient. The risk of sepsis increases with the employment of urinary drainage catheters, where normal flora, and/or bacteria from feces or skin easily ascend into the bladder around the inserted catheter.

In addition, residual urine in stasis around the retention balloon provides a culture medium at warm body temperatures that facilitates the growth of bacteria. Consequently, bacteria are able to accumulate, multiply and become pathogenic in the bladder, eventually circulating into the kidneys and throughout the system, resulting in sepsis of the system. Because of this propensity to produce infection in the patient, medical practitioners often refuse to extend the use of catheters, despite their usefulness.

Urinary tract infections (UTI's) are the most common nosocomial infection, and greater than 90% of these are catheter related (Nicolle (2001) Infections in Medicine, 18: 153; Sedor and Mulholland (1999) Urol Clin North Am, 26: 821). Nosocomial UTI's are a source of increased morbidity, mortality, and increasing financial burden of healthcare systems worldwide, accounting for more than than 1 million cases in U.S. hospitals annually (Foxman (2003) Dis Mon, 49: 53; Biering-Sorensen et al. (2001) Drugs, 61: 1275). Each episode of symptomatic nosocomial UTI adds nearly $700-1,500 dollars to the hospital bill (Saint (2000) Am J Infect Control, 28: 68), and an annual cost to the US healthcare system of nearly $451 million dollars (Jarvis (1996) Infect Control Hosp Epidemiol, 17: 552). Catheter-related bacteremia is estimated to cost nearly $2,900 per episode (Id.). Subpopulations at greatest risk for nosocomial catheter related UTI (the elderly, paraplegics, infants, pregnant women, diabetics, and patients with HIV/AIDS) (Id.).

The risk of UTI increases with increasing duration of catheterization. Recurrent infections lead to bacterial resistence to antibiotics. Long term catheterization has been associated with severe complications such as pyelonephritis (Warren (2001) Int J Antimicrob Agents, 17: 299; Huang et al. (2004) Infect Control Hosp Epidemiol, 25: 974), nephrolithiasis, epididymitis and prostatitis (Warren et al. (1994) J Am Geriatr Soc, 42: 1286). Bacteremia can occur when large static urine volumes and infection are combined with local urothelial trauma from chronic factors such as: catheter erosion, focal bladder wall ischemia due to persistent increased intraluminal pressures, and acute trauma from excessive catheter traction (Seiler and Stahelin (1988) Geriatrics, 43: 43). The discomfort associated with a distended bladder can caused unsupervised patients to pull their catheters out, resulting in urethral trauma/stricture, bleeding, and bacteremia.

Despite increasing numbers of patients with chronic indwelling Foley catheters, product innovation in this field has been limited to classes of material coatings designed to impede bacterial migration over the catheter and into the patient. Such new products have naturally focused on the urethral catheter component of the drainage system. For example, less reactive catheter materials such as silicone (Graiver et al. (1993) Biomaterials, 14: 465), low friction coatings such as Teflon, BN-74, and Hydrogel, and drug-eluting and silver impregnated surface coatings (Graiver et al. (1993) Biomaterials, 14: 465; Klarskov et al. (1986) Acta Obstet Gynecol Scand, 65: 295; Sabbuba et al. (2002) BJU Int, 89: 55; Gaonkar et al. (2003) Infect Control Hosp Epidemiol, 24: 506) were developed to decrease catheter-associated UTI's. These products have demonstrated inconclusive efficacy and unfavorable cost-effective value for even short-term prevention of of urinary tract infections. No practical advances in product design have been made to improve long-term urinary catheter-related tract infection rates.

While bacteriostatic/bacteriosidal materials coatings active at the level of the catheter make intuitive sense to help prevent nosocomial UTI's, but such measures are ineffectual when persistent residual volumes of urine within the bladder serve as a medium for bacteria and source of infection.

Blockage is problem frequently reported by more than half of outpatients with chronic urinary catheters (Wilde (2003) J Adv Nurs, 43: 254; Kunin et al. (1987) J Urol, 138: 899). The literature suggests that the most common causes of catheter blockage include blood clots, sediment crystals and mucus within the catheter lumen (Getliffe (1994) J Adv Nurs, 20: 140). Catheter blockage accounts for many unscheduled office, evening and weekend visits, in addition to emergency room visits and visits by home nurses (Wilde (2002) Home Healthc Nurse, 20: 449). A study examining after-hours home care nursing calls notes that 22 of 25 patients reported catheter-related problems (Wilde (2003) J Adv Nurs, 43: 254).

### SUMMARY OF THE INVENTION

In certain embodiments this invention provides systems for improved drainage from a bladder in a patient. The system typically comprises a fluid collection apparatus; a drainage receptacle; and a connecting tube comprising a means for reducing or eliminating airlocks in said connecting tube and thereby providing sufficiently low backpressure such that a patient having a urinary bladder drained with said system maintains an average residual bladder urine volume of less than about 50 cubic centimeters more preferably less than about 30 or 25 cubic centimeters over a period of at least four hours preferably at least 8 hours after initial drainage without manipulation of components of the system. In various embodiments the collection apparatus comprises a Foley catheter. In various embodiments the drainage receptacle comprises a urine collection bag, e.g., a vented urine collection bag.

In certain embodiments the means for reducing or eliminating airlocks comprises a means for producing a downward spiral shape in said connecting tube. Such means include, but are not limited to an external semi-rigid coil through which the connecting tube is threaded, an external semi-rigid coil to which said connecting tube is attached, and/or a semi-rigid coil formed from all or a part of said connecting tube. In various embodiments the means for reducing or eliminating airlocks comprises a tensioner attached to the connecting tube. Suitable tensioners include, but are not limited to a spring or elastic strap attached to the connecting tube, an elastic or elasticized bellows tubing, a form for wrapping escess collection tubing, and/or an auto-winder. In various embodiments the tensioner is removably attached to said connecting tube. In various embodiments the tensioner comprises a clip for attachment to bedding, a bedside, or an iv stand.

In certain embodiments this invention provides a medical drainage device or system comprising a connecting tube, where the connecting tube comprising a means for reducing or eliminating airlocks in the connecting tube and thereby providing sufficiently low backpressure such that when a patient is equipped with said device or system to drain a bladder, said patient maintains an average residual bladder urine volume of less than about 50 cubic centimeters over a period of at least four hours after initial drainage without manipulation of components of said system. In various embodiments the means for reducing or eliminating airlocks comprises a means for producing a downward spiral shape in said connecting tube. In certain embodiments such means include, but are not limited to an external semi-rigid coil through which the connecting tube is threaded, an external semi-rigid coil to which said connecting tube is attached, and/or a semi-rigid coil formed from all or a part of said connecting tube. In various embodiments the means for reducing or eliminating airlocks comprises a tensioner attached to the connecting tube. Suitable tensioners include, but are not limited to a spring or elastic strap attached to the connecting tube, an elastic or elasticized bellows tubing, a form for wrapping escess collection tubing, and/or an auto-winder. In various embodiments the tensioner is removably attached to said connecting tube. In various embodiments the tensioner comprises a clip for attachment to bedding, a bedside, or an iv stand. In various embodiments the device or system further comprises a drainage device selected from the group consisting of a urinary catheter, a chest tube, a mediastinal tube, a nasogastric tube, and/or a Jackson Pratt tube. In various embodiments the drainage device or system further comprises a urinary leg bag which can optionally comprise a venting tube.

This invention also provides a urinary catheter comprising a connecting tube, where the connecting tube comprises a downward spiral shape.

Also provided is a urinary leg bag comprising a connecting tube and a venting system, wherein said connecting tube comprises a downward spiral shape and said venting system comprises a venting tube.

In certain embodiments this invention provides a collecting tube for use in a medical drainage device, where the collecting tube comprises a means for reducing or eliminating airlocks in the tube. In certain embodiments the means for reducing or eliminating airlocks comprises a means for producing a downward spiral shape in said connecting tube. Such means include, but are not limited to an external semi-rigid coil through which the connecting tube is threaded, an external semi-rigid coil to which said connecting tube is attached, and/or a semi-rigid coil formed from all or a part of said connecting tube. In various embodiments the means for reducing or eliminating airlocks comprises a tensioner attached to the connecting tube. Suitable tensioners include, but are not limited to a spring or elastic strap attached to the connecting tube, an elastic or elasticized bellows tubing, a form for wrapping escess collection tubing, and/or an auto-winder. In various embodiments the tensioner is removably attached to said connecting tube. In various embodiments the tensioner comprises a clip for attachment to bedding, a bedside, or an iv stand. In certain embodiments the tube comprises means for attachment to a waste receptacle for biological fluids.

This invention also provides methods of reducing urinary tract infection in a subject bearing a urinary catheter. The method typically involves providing a connecting tube coupled to the catheter where said a connecting tube comprises a means for reducing or eliminating airlocks in said connecting tube and thereby providing sufficiently low backpressure such that a patient having a urinary bladder drained with said system maintains an average residual bladder urine volume of less than about 50 cubic centimeters over a period of at least four hours after initial drainage without manipulation of components of said system. In various embodiments the catheter is a Foley catheter. In various embodiments the means for reducing or eliminating airlocks comprises a means for producing a downward spiral shape in the connecting tube. Such means include, but are not limited to an external semi-rigid coil through which the connecting tube is threaded, an external semi-rigid coil to which said connecting tube is attached, and/or a semi-rigid coil formed from all or a part of said connecting tube. In various embodiments the means for reducing or eliminating airlocks comprises a tensioner attached to the connecting tube. Suitable tensioners include, but are not limited to a spring or elastic strap attached to the connecting tube, an elastic or elasticized bellows tubing, a form for wrapping escess collection tubing, and/or an auto-winder. In various embodiments the tensioner is removably attached to said connecting tube. In various embodiments the tensioner comprises a clip for attachment to bedding, a bedside, or an iv stand.

In certain embodiments the connecting tube is not a straight tube.

Also provided are kits for draining a biological fluid from a site in a subject. The kits typically comprise a a collecting means for application to the site; and a connecting tube comprising a means for reducing or eliminating airlocks in said connecting tube and thereby providing sufficiently low backpressure such that a patient having a urinary bladder drained with said connecting tube maintains an average residual bladder urine volume of less than about 50 cubic centimeters over a period of at least four hours after initial drainage without manipulation of components of said system, e.g., as described herein. In various embodiments the kits further comprising a waste receptacle for receiving biological fluid drained from said site. In various embodiments the collecting means is a Foley catheter, a Jackson Pratt tube, and/or a nasogastric tube. In certain embodiments the kits further comprise instructional materials teaching the use of the drainage device with the collecting tube.

In certain embodiments this invention provides an iv stand comprising a container or support for containing or holding a waste receptacle for biological fluids. In various embodiments the iv stand is on wheels.

### DEFINITIONS

A “biological fluid” refers to any one or more fluids produced by a biological organism. Such biological fluids include, but are not limited to urine, seminal fluid, cerebral spinal fluid, blood or blood fractions, plasma, saliva or other oral fluid, stomach fluid, bile, pus, liquefied tissues, and the like.

A “connecting tube” or “collecting tube” refers to the tubing of a medical drainage system, connecting a fluid collection device (e.g., a catheter) and a waste receptacle (e.g., urine collection bag).

The term “downward spiral shape” refers to a spiral shape that is downward from the patient to the collection bag; net fluid flow in a downward spiral shape is down.

A “medical drainage device” or “medical drainage system” refers to any drainage apparatus for collection of a biological fluid. Such devices include, but are not limited to, a Foley catheter, any other catheter, a nasogastric tube, a mediastinal tube, a urinary leg bag, and the like. In certain embodiments a medical drainage system comprises a fluid collection device (e.g., a catheter), a waste receptacle, and a connecting tube or connecting tube linking the fluid collecting device to the waste receptacle.

A “tensioner” refers to a device that provides tension to a connecting tubing and thereby removed or reduces dependent loops.

An “auto-winder” refers to a device that winds up a connecting tubing and can thereby act as a tensioner.

### BRIEF DESCRIPTION OF THE DRAWINGS

These and other features, aspects, and advantages of the present invention will become better understood with regard to the following description, and accompanying drawings, where:

FIG. 1 illustrates the formation of a airlock in a typical urinary drainage system. The drainage receptacle 16 is typically hung such that the connecting tube 14 forms a dependent loop 21 thereby forming an airlock.

FIG. 2 schematically illustrates a medical drainage system comprising a collection apparatus 12, a drainage receptacle 16 having a vent 15 and hangers 19, and a connecting tube 14 comprising a downward spiral section 13.

FIG. 3 is a diagram of a connecting tube affixed to a semi-rigid adjustable coil. The coil comprises a semi-rigid adjustable coiled backbone 33 attached to a number of rings or clasps 35. The connecting tube 14 is attached to the clips or threaded through the rings to provide an effective downward spiral 37.

FIG. 4 schematically illustrates a spiral produced in connecting tube 14 by an external semi-rigid tube 39.

FIG. 5 illustrates a connecting tube 14 having an intrinsic downward spiral. The interior portion of the tube is manufactured with a narrower trough shape 5 throughout to ensure flow of even minimal amounts of urine.

FIG. 6 illustrates a of a urinary collection tube affixed to a semi-rigid adjustable coil.

FIGS. 7A and 7B illustrate a simple spring or elastic tensioner 23 attached to the connecting tube 14 to prevent the formation of a dependent loop.

FIG. 8 illustrates a tensioner comprising a bellows region 18 compressed by spring or elastic elements 17.

FIGS. 9A, 9B, and 9C illustrate an autowinder for providing continuous tension on a connecting tubing 14.

FIG. 10 illustrates an IV pole 61 fitted with a canister 66 for containing a drainage receptacle 16 coupled to a connecting tube 14. The IV pole comprises a hook 62 for supporting an IV bag and can optionally comprise wheels 65.

FIG. 11 illustrates an IV pole 61 fitted with a hanger 64 for supporting a drainage receptacle 16 coupled to a connecting tube 14. The IV pole comprises a hook 62 for supporting an IV bag and can optionally comprise wheels 65.

FIG. 12 shows the model system with the basin filled with water and the outflow clamped. This schematic represents the full bladder immediately before catheterization.

FIG. 13 shows the clamp on the Foley released, allowing drainage though the system. If the bag is located below the level of the basin, the basin will drain to completion due to a “siphoning effect” from the tubing. This schematic represents the initially catheterized bladder. Note the absence of any fluid within the tubing before the clamp is released to allow flow.

FIG. 14 illustrates the model system where the basin was prevented from draining to completion (by applying the clamp to the Foley) because this would allow air into the Foley catheter from above. In-vivo, air does not normally enter the Foley from the bladder, since the bladder is a closed space. After clamping the Foley, the fluid within the drainage tubing is then “milked” distally, as would normally occur by nursing staff or whenever the patient ambulates.

FIG. 15. As the clamp is slowly released, a trickle of fluid then enters the tubing (to simulate production of urine after catheterization). This fluid collects in the dependent-most portion of the curled tubing, and the rising fluid level soon occludes the lumen of the tube. When the lumen of the drainage tube is occluded, the air within the proximal segment of the drainage tubing becomes “trapped”. Pressure within this segment rises as newly produced urine tries to follow gravity to flow downward. Pressure within the distal segment is always equal to atmospheric pressure, due to the air vent within the bag.

FIG. 16 illustrates the arrangement of urinary catheter/drainage tubing as typically observed at bedside. Note the curled dependent segment of drainage tubing. This segment usually lies just proximal to the bag because the is hung from the bed by hooks. Redundant tubing falls below the level of the bag, causing the “curl”. Note that the menisci are asymmetric in height, suggesting that a pressure differential exists across the drainage tube.

FIG. 17. A standard 18 Fr. Foley catheter and drainage tubing kit is used to drain a 100 cm tall column of water. Because the pressure within the proximalmost segment of drainage tubing equals the pressure within the bladder, a pressure sensor located in this segment is a surrogate for bladder pressure. An air-lock is created by repeating the sequence of steps in FIGS. 13-16 while maintaining a low fluid level within the column. Once the air-lock is established, water is dribbled into the column. As this occurs, the pressure within the column rises, and the distal meniscus rises toward the apex of the upper curl.

FIG. 18. We recorded the pressure at the exact moment the distal meniscus reached the apex of the upper curl to establish drainage into the collection bag. This pressure equals the maximum obstruction pressure for the air-lock generated by curling the tubing to that particular height. The magnitude of the obstruction pressure (cm H₂O) is proportional to the height (cm).

FIG. 19 shows a tubing system with downward-spiral coiled conformation.

FIG. 20 shows the results of pressure x flow trials using the novel coiled tubing system. The coiled conformation of the tubing precludes the presence of dependent areas within the tubing in which fluid stasis can occur.

FIG. 21 shows a variation of proposed the coiled tubing system, utilizing a shorter tubing length to minimize the likelihood that the tubing will be inadvertently curled at bedside.

FIG. 22 shows a detailed schematic illustrating the mathematical basis of air-locks.

### DETAILED DESCRIPTION

Medical Drainage Devices and Systems.

Medical drainage devices are commonly used to drain biological fluids from patients. Examples of medical drainage devices are urinary catheters for drainage of urine; nasogastric tubes for drainage of gastric fluid, mediastinal tubes for drainage of residual irrigating solutions and blood, a Jackson Pratt Drain or “JP” (bulb drain), for draining a wound, and the like.

In various embodiments a medical drainage device or system typically includes a fluid collection apparatus (e.g., a catheter) positioned at the site in the patient from which fluid is to be collected, a drainage receptacle (e.g., a waste receptacle) into which the collected fluid is disposed, and a collection or connecting tube joining the fluid collection apparatus to the waste receptacle. The connecting tube generally has a variable length to accommodate the varying distance between the patient and the connecting tube, depending on the location of the patient.

It has been observed by the instant inventors that medical drainage devices do not drain biological fluids consistently nor optimally. For example, a urinary drainage appliance typically comprises three elements: a urinary catheter, typically at “Foley catheter,” a connecting tube and a collection bag. Clinical observations by the inventors have measured residual bladder urine volumes of 400-500 cc in patients with such a urinary drainage appliance in place. Such residual bladder urine volumes can adversely affect anastomatic tissue healing and facilitate urinary extravasation. In addition, the residual urine can increase the likelihood of noscomial urinary tract infection and thus suboptimally drain the upper urinary tracts.

Without being bound by a particular theory, it is believed that the suboptimal drainage of medical drainage devices, e.g., urinary catheters, is the result of airlocks that form within the connecting tube. Due to the variable length of the connecting tube, curls form in the connecting tube. These curls promote focal fluid stasis. These sites of fluid stasis lead to airlocks, which decrease the flow of biological fluid from the patient to the drainage reservoir.

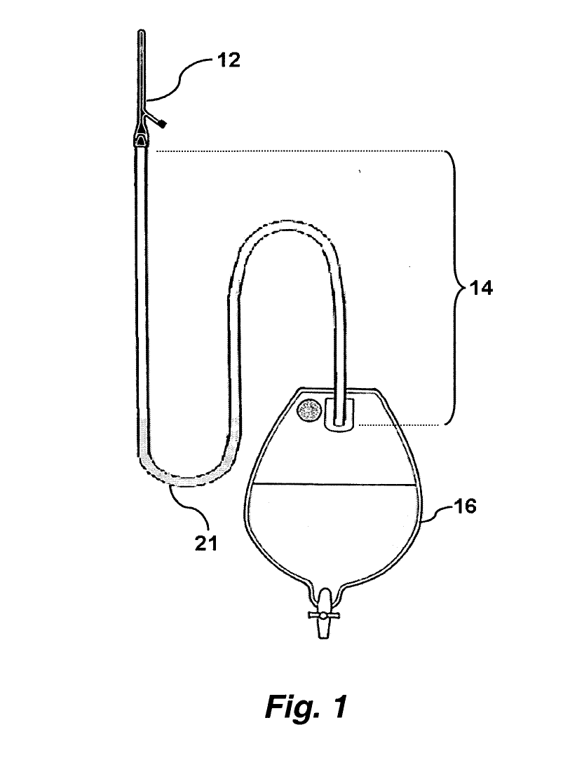

This is schematically illustrated in FIG. 1 which shows the typical disposition of a urinary drainage system. The collection apparatus 12 (e.g. a Foley catheter) is coupled to a drainage receptacle 16 by a connecting tube 14. In typical instances, the connecting but 14 and drainage receptacle 16 are disposed such that the connecting tube 14 forms a dependent loop 21 which forms an “airlock” thereby increasing the pressure required to drain the bladder. After the initial drainage associated with insertion of the drainage system into the patient, the airlock forms resulting in incomplete drainage of the bladder. Without being bound to a particular theory, it is believed the failure to adequately drain the bladder leads to a significant increase in urinary tract infections.

Thus, in certain embodiments, this invention contemplates the use of a connecting tube in various drainage applications, where the connecting tube comprises a means for reducing or eliminating airlocks in the tube and thereby providing sufficiently low backpressure to ensure effective drainage of the biological site and thereby reduce infection.

Accordingly, in certain embodiments, this invention provides a system for improved drainage from a bladder in a patient, where the system comprises: a fluid collection apparatus; a drainage receptacle; and a connecting tube comprising a means for reducing or eliminating airlocks in the connecting tube and thereby providing sufficiently low backpressure such that a patient having a urinary bladder drained by the system maintains an average residual bladder urine volume of less than about 100 cubic centimeters, preferably less than about 50 cubic centimeters, and more preferably less than about 25 cubic centimeters over a period of at least four hours, more preferably at least 6 hours, and most preferably at least 8, 10, 12, or 24 hours after initial drainage without manipulation of components of the system.

As indicated above, in certain embodiments present invention reduces the number of airlocks in the connecting tube. By reducing the number of airlocks, the flow of biological fluids from the patient to the collection bag increases, thereby improving drainage and tissue healing and decreasing risk of infection.

Any number of different means can be used for reducing the number of airlocks in a medical drainage device, thereby increasing drainage. In one embodiment, reducing the number of airlocks in the connecting tube is accomplished by incorporating a downward spiral shape into the connecting tube.

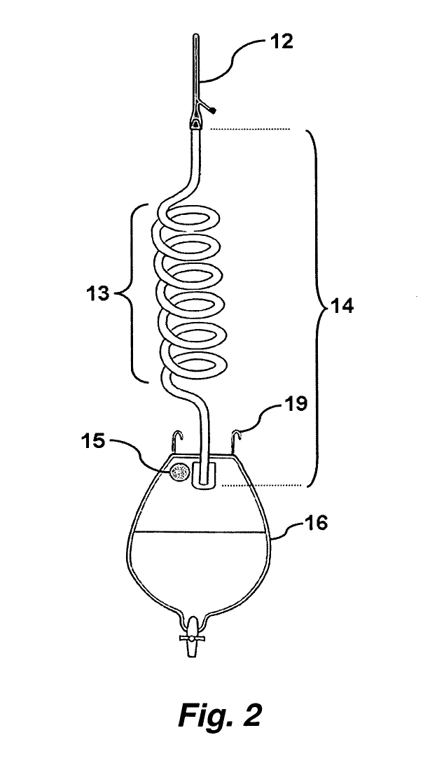

One example of such a downward spiral is illustrated in FIG. 2. This figure schematically illustrates a medical drainage system comprising a collection apparatus 12, a drainage receptacle 16 having a vent 15 and hangers 19, and a connecting tube 14 comprising a downward spiral section 13. The downward spiral shape of the connecting tube overcomes the curls of a standard connecting tube that are responsible for the airlocks. The downward spiral shape is flexible, e.g., bendable, and extendible, to allow use while a patient is either bed bound, or during ambulation.

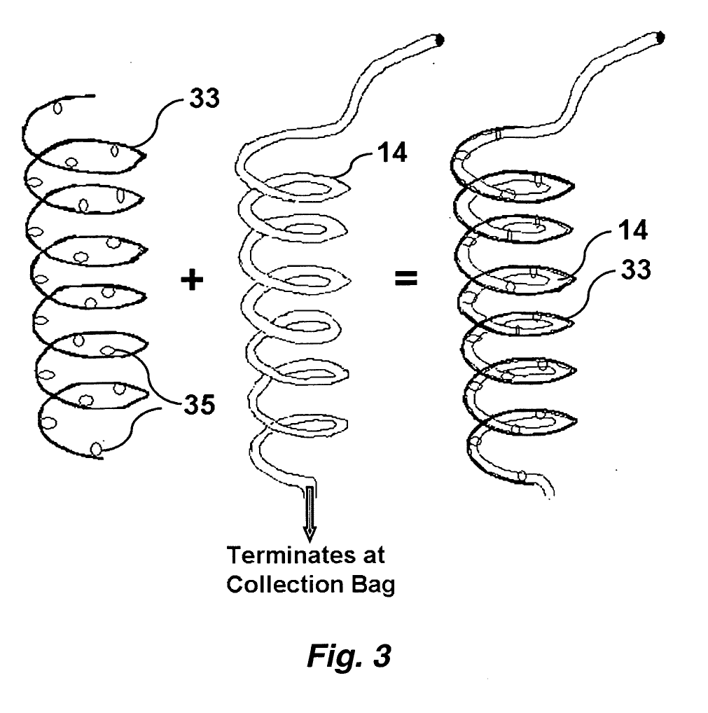

In certain embodiments of the invention, the downward spiral shape is accomplished by affixing the connecting tube to an external semi-rigid coil that shaped in the downward spiral shape (see, e.g., FIG. 3). An external semi-rigid coil 33 can be made from any number of materials, including, e.g., any material that is flexible, e.g., metal or plastic. In one embodiment, the external semi-rigid coil is made from one quarter inch hollow copper (or other metal) or plastic tubing. In some embodiments the external semi-rigid coil is reusable and can be sterilized, e.g., autoclaved. In other embodiments, the external semi-rigid coil is disposable.

The connecting tube 14 can be affixed to the external semi-rigid coil 33 in any number of ways, including, but not limited to rings 35, clasps, snaps, Velcro, tape, and the like. The semi-rigid coil 33 has a downward spiral shape so that affixing the connecting tube 14 to the semi-rigid coil 33 gives the connecting tube a similar downward spiral shape. Net fluid flow through the connecting tube is down, e.g. from the patient to the collection bag.

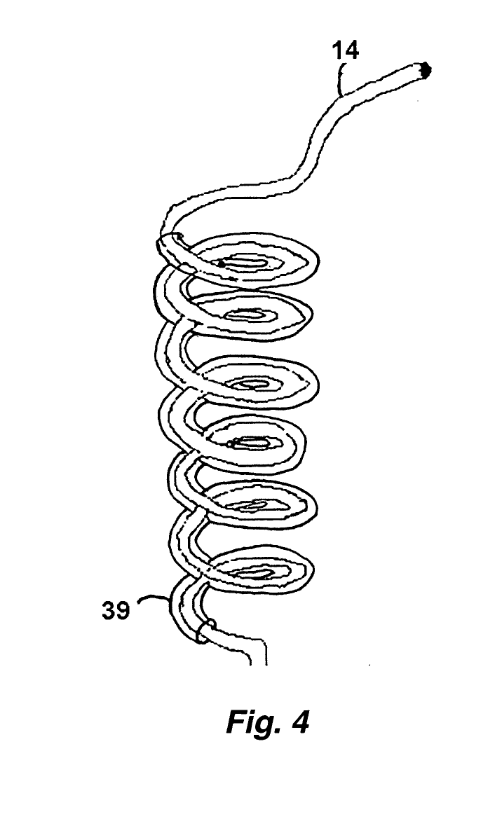

In certain embodiments the downward spiral shape of the connecting tube 14 is provided by an external semi-rigid spiraled tube (exoskeleton) 39, e.g. as illustrated in FIG. 4. In some embodiments the external semi-rigid tube 39 is reusable and can be sterilized, e.g., autoclaved. In other embodiments, the external semi-rigid tube 39 is disposable.

In certain embodiments the connecting tube 14 itself is manufactured in a downward spiral shape (see, e.g., FIG. 2).

In various embodiments the connecting tube 14 can be manufactured with a narrower, trough shape on the inferior portion of the tubing to ensure flow of even minimal amounts of biological fluids.

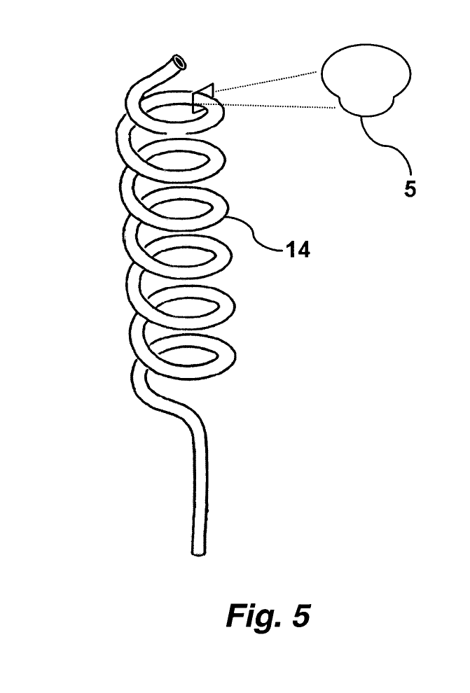

Another example of a connecting tube of the invention is shown in FIG. 5. Here the connecting tube 14 is manufactured with a downward spiral shape. In addition, the connecting tube can be manufactured with a trough 5, shown in the cross section.

In various embodiments the invention includes medical drainage devices that include a connecting tube with a downward spiral shape as described herein. Illustrative medical drainage devices include but are not limited to a urinary catheter, e.g., a Foley catheter, a nasogastric tube, a mediastinal tube, a urinary leg bag, a Jackson Pratt tube, and the like.

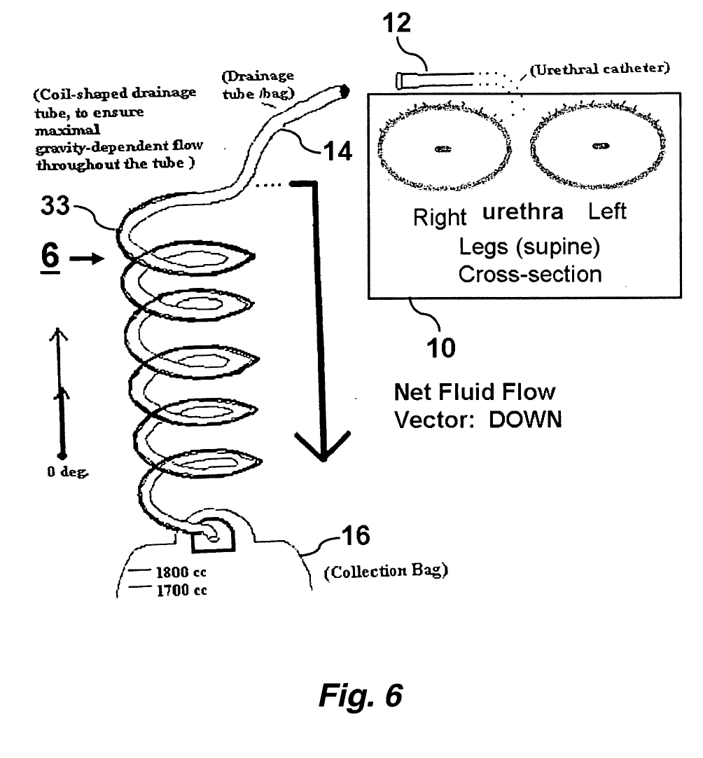

In one variation, the invention is a urinary drainage appliance that includes a connecting tube in a downward spiral shape. One example is illustrated in FIG. 6. The urinary drainage appliance 6 includes the collection bag 16 and the connecting tube 14. The connecting tube 14 is attached to a semi-rigid coil 33 of, e.g. one quarter inch hollow copper or plastic tubing. Urine flows from the patient 10 down through the connecting tube to the collection bag. The flow path is devoid of regions require the fluid to flow upward and so minimizes airlocks and promotes fluid drainage.

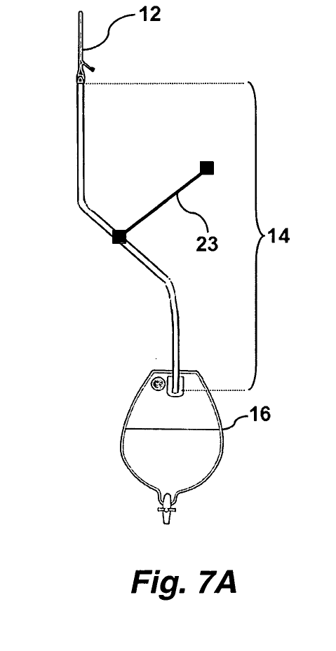

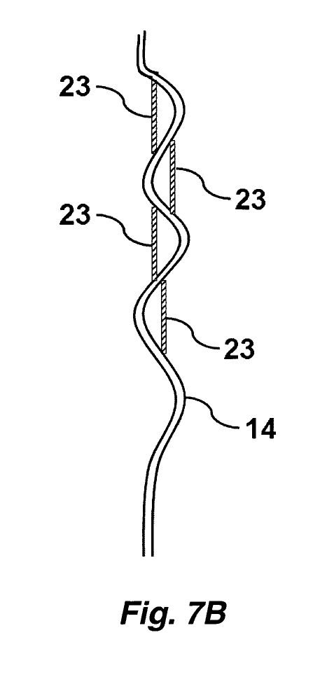

In certain embodiments, the means for reducing airlocks in the collecting tube can comprise various tensioners or autowinders. A tensioner acts to provide a tension along the connecting tube 14 generally upwards away from the drainage receptacle 16 and thereby resists the formation of a dependent loop (e.g., 21 in FIG. 1). Any of a variety of tensioners are well suited to use in the present invention. In one very simple embodiment, the tensioner comprises an elastic strip or spring. Thus, for example, FIG. 7A illustrates the use of a simple strap, elastic, or spring to eliminate/prevent dependent loop formation. The strap, elastic or spring can have one clip that attaches to the connecting tube 14 and another clip that attaches to the bedside, bedding or other convenient location.

FIG. 7A illustrates another embodiment where a simple elastic or spring is attached to the connecting tube 14 at multiple points.

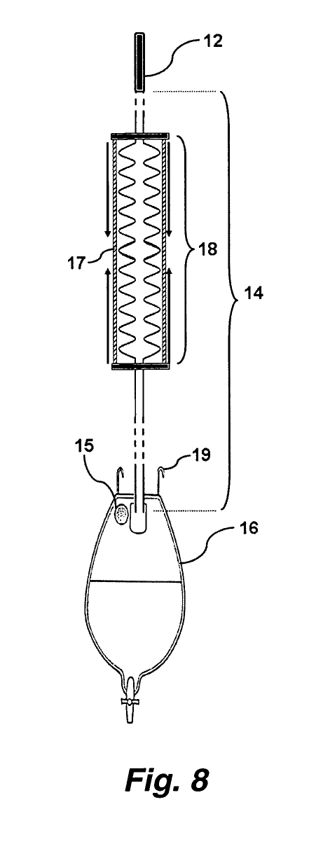

Another tensioner comprises a bellows arrangement, e.g., as illustrated in FIG. 8. Here the connecting tube 14 comprises a bellows region 18 compressed by spring or elastic elements 17. The bellows allows lengthening of the connecting tube 14 while the elastic elements 17 prevent the formation of dependent loops.

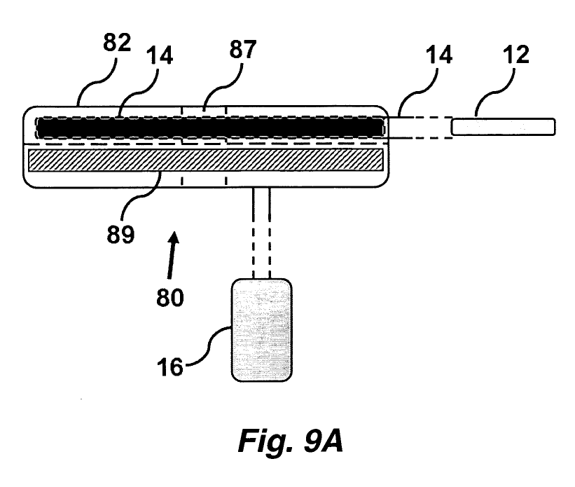

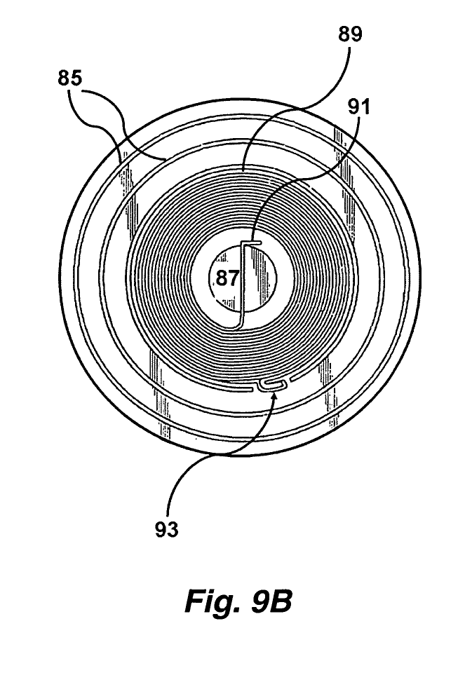

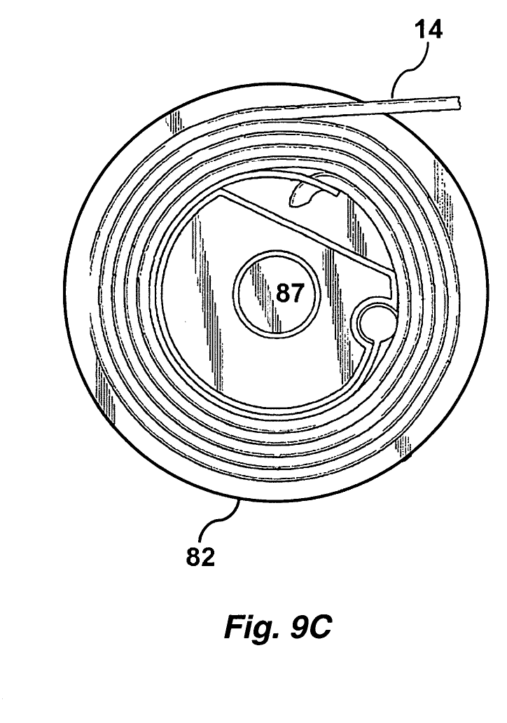

In various embodiments the connecting tube 14 can be provided with an auto-winder. The auto-winder continually coils up slack tubing thereby preventing the formation of dependent loops. One auto-winder is illustrated schematically in FIGS. 9A, 9B, and 9C. As illustrated therein, the autowinder 80 can comprise a winder housing 82 bearing a central spool 87. One side of the housing comprises a coiled spring 89 (see, e.g., FIGS. 9A and 9B), while the other side of the housing comprises the collecting tube wrapped around the spool (see, e.g., FIGS. 9A and 9C).

The coiled spring comprises an internal tab 91 attached to the spool 87 and an external tab 93 attached to the housing 82. When “wound up” the spring tends to cause the spool 87 to rotate thereby wrapping the tubing 14 around the spool. The bottom surface additionally includes a series of circumferential rings or ridges 85 that serve as bearings for the rotating spool 87 against the flat lower surface of the housing cavity.

The connecting tubing 14 can be extended from the autowinder 80 as needed to connect the collecting apparatus 12 with the drainage receptacle 16. Drawing of the tubing 14 out of the winder tensions the spring 89 which tends to rewind the tubing thereby tensioning the tubing and preventing formation of dependent loops. The autowinder of FIGS. 9A-9C is intended to be illustrative and not limiting.

Other means for preventing the formation of airlocks include an adjustable “arm” unit that can be clamped to the bed or IV pole, etc, which serves to hold-up the connecting tubing 14 to a height, again, ˜3 feet high (approx half the bed height). Also, it serves to hold the tubing away from the patient's bed, effectively reducing the tubing length available to become curled in a dependent manner, etc. In various embodiments the armature can include a flexible metal or plastic arm (like adjustable desk lamps) that has a clamp, bracket, etc on one end (to fix it to the bed, IV pole, bedside table, etc), and the other end is designed to hold the drainage tubing: it can be form-fitting to the tubing, and secured with a velcro strap, plastic snap clamp, snap-locking circumferential fitting, etc). The angle of the clamp, relative to the arm, can be made adjustable if the clamp's connection point to the arm has a swivel design that can be tightened to secure any desired orientation.

Another device that serves the same purpose is a “drape” that is hung over the side of the bed with the Foley. In various embodiments this drape has a velcro backing, and velcro straps, that can be placed anywhere, so that the Foley tubing is simply run along the drape (toward the foot of the bed), and the velcro straps are placed over the tubing, to secure it in a linear and net downward orientation. The drape can be also fitted with rings anywhere, or in columns every 6 inches, so that the drainage receptacle can be hung from the drape virtually anywhere.

The foregoing means of limiting or preventing airlocks in the connecting tube 14 are intended to be illustrative and not limiting. Using the teaching provided herein, other modifications can be provided with at most routine modification.

Drainage Receptacle Hangers and Containers.

In various embodiments this invention also provides novel draingage receptoacle hangers and containers. Thus, for example, in certain embodiments, this invention provides a “uro-receptacle” for bedside use to maintain the connecting tubing 14 in a non-dependent downward orientation. In certain embodiments the “uro-receptacle” comprises a container that is ˜3 feet in height (typically less than the average bed height but preferably at least as high as half the height from floor to bed), shaped to accommodate the connecting tube. Thus, for example, in certain embodiments, the uro-receptacle can comprise e.g. a 3 foot canister with a smooth/curved spigot shape at one corner, through or over which the connecting tube passes toward the drainage receptacle 16 at the bottom of the uro-receptacle.

In certain embodiments the canister can be fitted with a spigot whose height on the canister is adjustable, thereby allowing the raised height of the connecting tube to be adjusted as needed (for example, ICU beds are typically higher from the ground than normal ward beds, and therefore, the spigot could be lowered or raised, as needed.

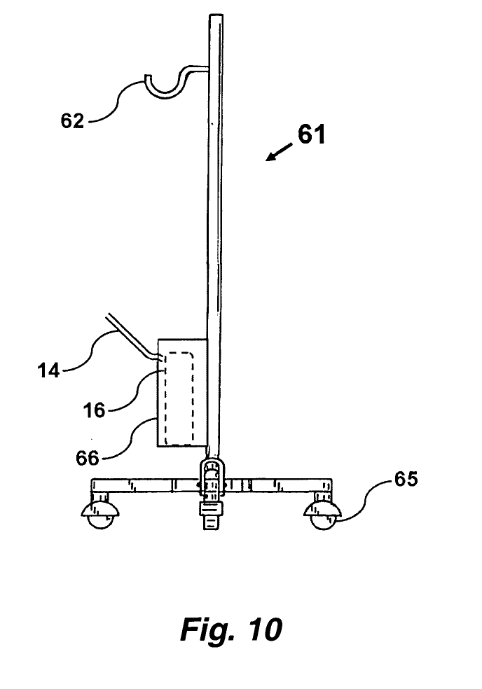

The same canister can be fitted with wheels or it can be attached to an IV pole, for ambulatory patients (see, e.g., FIG. 10).

Also for ambulatory patients, in certain embodiments, this invention provides a uro-receptacle that, on the inside, comprises a hard plastic spiral, to which snap the connecting tubing 14 can be attached to enforce a downward, non-dependant trajectory of urine along the tube.

In addition to maintaining optimal drainage during ambulation (when patients typically carry their bags, thus creating a huge dependant curl), this device also serves a second, “novel” purpose: because the portable version of the uroreceptacle can be made of colored plastic, it offers some element of privacy, by obscuring the nature of the bag contents.

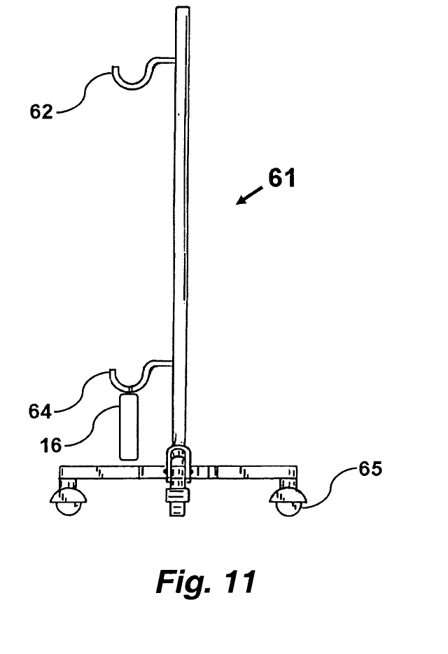

In certain embodiments this inventnion also provides an IV pole comprising a second hanger for attaching a drainage receptacle 16 (see, e.g., FIG. 11).

Urinary Leg Bags.

Urinary leg bags are smaller urine collection bags that can be attached with straps to the leg, and therefore be worn under regular clothing, so as to allow more discrete use of the urinary catheter, in public. It has been observed that urinary leg bags demonstrate suboptimal drainage characteristics. Urine draining into the bag, does not appear to drain freely, and is observed to collect within the drainage tubing leading to the bag.

In a standard bedside urinary collection bag, there is a ˜1 inch reound venting patch of permeable plastic material at the top-most area of the bag this material is generally woven plastic, and allows air to pass through in either direction. This venting system exposes the lumen of the urinary collection bag to atmospheric pressure at any filling volume.

Urinary leg bags cannot contain such a venting patch. Urinary leg ags are exposed to extrinsic mechanical pressure (the wearer's leg movement with ambulation/bending over, etc.) that can cause the fluid level to rise that can cause the fluid level to rise within the bag, and result in leakage the moment the fluid level rises to the “venting patch”. The result is that a standard urinary leg bag demonstrates resistance to inward flow of fluid. The volume occupied by air trapped within the partially empty bag and the empty connecting tube, e.g., the airlock, cannot be completely displaced by incoming urine, since it is a closed system. Therefore, there is resistance to antegrade flow of urine from the bladder into the bag.

It would be beneficial to have urinary leg bag that overcomes this airlock, e.g., a urinary leg bag that includes a venting sytem for improved drainage. Accordingly, one embodiment of the invention is a urinary leg bag with improved drainage using a venting system.

In one embodiment the collection bag venting system comprises a venting tube. One end of the venting tube connects the top of the collection bag. The venting tube can extend along the entire length of the connecting tube to about the level where the connecting tube connects to the patient, e.g, with the Foley catheter. The second end of the venting tube can be exposed to air.

The venting tube element can be attached to the collection tube via, e.g, clasps. Alternatively, the venting tube element is permanently attached to the connecting tubing, e.g., the venting tube is glued to the connecting tube. In certain embodiments, the collecting tube is manufactured with the venting tube affixed.

In some embodiments, the second end of the venting tube is capped, e.g., plugged with a permeable material, e.g., cotton, sponge, etc. The permeable material allows air flow through the venting tube. In addition, the permeable material is absorbent to minimize spillage of biological fluid, e.g., urine. IN further embodiments the permeable material can be replaced if wet.

In some embodiments, the venting tube includes a second lumen within the first, e.g., a tube within a tube. Both lumens of this venting tube are connected to the collection bag at one end, and are exposed to air at the second end near where the collection tube enters the patient. The inner lumen is shorter than the outer lumen. If fluid, e.g,. urine, rises throught he iner tube from the collection bag, the fluid trains into the larger diameter outer tube and returns to the collection bag.

In some embodiments, the venting tube includes a fluid trap. In one variation of this embodiment, the venting tube runs horizontally for a short distance to create a fluid trap. Any fluid, e.g., urine that refluxes from the collection bag across this horizontal portion falls into the fluid trap. Biological fluid in the fluid trap is returned to the collection bag.

While the invention has been particularly shown and described with reference to a preferred embodiment and various alternate embodiments, it will be understood by persons skilled in the relevant art that various changes in form and details can be made therein without departing from the spirit and scope of the invention.

### EXAMPLES

The following examples are offered to illustrate, but not to limit the claimed invention.

### Example 1

Suboptimal Bladder Drainage with Traditional Foley Drainage Systems: a Better Way

Objective

Foley catheters are assumed to completely drain the urinary bladder. We have routinely observed evacuation of large retained urine volumes upon manipulation of the drainage tubing. Drainage characteristics of Foley catheter systems are poorly understood. To investigate unrecognized retained urine with Foley catheter drainage systems, we measured bladder volumes of hospitalized patients with standard bladder-scan ultrasound volumetrics. Additionally, an in-vitro benchtop mock bladder and urinary catheter system was developed to understand the etiology of such residual volumes. Based on these findings, we designed and tested a novel drainage tube design that optimizes indwelling cathteter drainage.

Methods:

Bedside bladder ultrasound volumetric studies with a 3.5 mHZ transducer (Diagnotic Ultrasound, USA) were performed on hospitalized ward and ICU patients in the early morning. If ultrasound noted the presence of residual urine, the drainage tubing was manipulated in an up-down fashion to facilitate drainage. An ex-vivo bladder-urinary catheter system was made using a 6-L. flat-bottomed basin drained with an 18 Fr. Foley catheter inserted at its base. The catheter and drainage bag/tubing (BARD, USA) were positioned in the same orientation (angles/ curvatures and distance of all segments to the floor) with a dependent curl proximal to the drainage bag. We performed trials to measure flow rates and pressures within the drainage tubing. To mimic the naturally closed-system urinary tract, we first filled the reservoir and commenced to drain the system. The dependant portion of the curled tubing segment was full of fluid to ensure that there would be no outlet for the air trapped between the curl and the reservoir. Flow trials were also performed using our novel drainage tube system.

Results:

150 patients underwent bladder ultrasound volumetrics (75 men; 75 women). In ICU patients (M=39/F=36), where urine output was measured hourly and where nursing staff routinely manipulate the Foley drainage tubing to maximize recorded urine output, mean residual volume was 96 ml (Range: 4-290 ml). In hospital ward patients (M=38/F=37), with nursing checks every 4 hours, mean residual volume was 136 ml (Range: 22-647 ml).

Laboratory Studies: Fluid collected within the dependant drainage tubing curl after initiation of reservoir evacuation. Once fluid completely filled the dependent portion of the curl, pressure proximal to the curl increased and drainage ceased. We observed that the pressure (cm H2O) within the proximal segment of drainage tubing equaled the height of the curled tubing (cm). For every 1 cm in curl height obstruction pressure would increase by 1 cm H2O. In contrast, our spiral-shaped drainage tube demonstrated rapid (0.5 cc/sec), continuous, and complete (100%) reservoir drainage in all trials.

Conclusions

Foley catheter drainage systems evacuate the bladder suboptimally. Outflow obstruction is caused by “airlocks” that invariably develop within curled redundant drainage tubing segments. Our novel drainage tubing design eliminates redundant, gravity dependent curls with their associated air-locks, optimizing flow and minimzing residual bladder urine.

Details

It is commonly assumed that a Foley catheter drainage system continuously and completely empties the urinary bladder. Our bedside observations suggest otherwise. We have routinely observed that the heights of the Foley drainage tube menisci are asymmetric, suggesting a pressure differential across the tube. Furthermore, upon routine manipulation of the drainage tubing during morning rounds (to inspect for clots, assess urine color, etc.), we have frequently observed sudden drainage of large volumes of urine. Such residual volumes are more commonly observed when the patient has been bedbound for at least 2 hours, and occur in the absence of obvious sources of obstruction such as blood clots, urine sediment, or kinking of the drainage tubing. Interestingly, catheterized patients often describe intermittent periods of the sensation of “bladder fullness”, especially after remaining in bed for long periods. Such complaints often resolve immediately after simple up/down manipulation of the drainage tubing to effect drainage of all visible urine into the bag. Again, manipulation of the drainage tubing causes immediate evacuation of large urine volumes, and the patient often reports the sensation of bladder evacuation.

We sought to compare and validate our observations with those of the nursing staff at our center. Nurses report that, routinely, 10-80% of the final recorded urine output is urine drained in response to drainage tube manipulation. Furthermore, nurses at our institution report that in nursing school, to maximize the accuracy of recorded urine outputs, they are taught to “milk” the drainage tubing before measuring the final output.

These observations suggest that appreciable undrained bladder residual volumes are possible, that they appear to occur after protracted periods of recumbancy, and that such residual volumes are somehow mobilized by simple manipulation of the drainage tubing. In sum, Foley urinary catheter systems do not drain the bladder efficiently or completely.

To test our hypothesis that bladder urine residual volumes occur in the absence of obvious causes of obstruction, we used a bedside bladder-ultrasound device to measure early morning mean bladder urine volume in patients with indwelling urinary catheters.

To better understand the mechanism of urinary drainage, we then created a urinary bladder model which we drained with a standard 18 Fr Foley catheters and drainage kit in order to reproduce the state of the catheter and drainage pattern observed at bedside. In order to replicate the state of a catheter system (from initial placement of the catheter to routine manipulation and emptying of the bag) as observed in vivo, we took into account variables such as known bladder and tubing urine volumes and pressures, the positioning of the catheter and drainage tubing/bag, curves in the drainage tubing, and relative heights and distances from the bladder to the drainage bag. We then performed controlled flow/pressure trials to quantify the bladder pressures associated with obstruction within the drainage tube.

Finally, based on our new insights, we designed and tested a novel drainage tubing system that we feel drains the bladder more completely and efficiently than the traditional system commonly used today.

Materials and Methods:

Bedside Bladder Ultrasound Volumes in Patients with Indwelling Catheters

After obtaining approval from our Committee on Human Research, 150 hospitalized patients (75 men, 75 women) with indwelling Foley catheters were enrolled. Half of the patients were in the Intensive Care Unit (ICU) (M=39/F=36) and half were in the regular hospital ward (M=38/F=37). Inclusion criteria included the presence of an indwelling Foley urinary catheter for at least 24 hours prior to ultrasound bladder volumetrics, visbily clear urine within the drainage tubing (free of visible sources of potential obstruction, such as clood clots, sediment/tissue, or calculi), absence of any obvious extrinsic cause of drainage tubing obstruction such as a kink in the tubing or bag, and recorded mean urinary output of at least 20 cc/hour within 12 hours of evaluation.

Patients who did not meet these criteria were excluded. In addition, patients were excluded if they had a suprapubic tube in place, unstable renal function, bandaged post-surgical suprapubic incisions or any anatomic deformity that precluded appropriate suprapubic access with an ultrasound probe for bladder volumetrics.

Trans-abdominal bladder volumetric ultrasonography was performed with a 3.5 mHz transducer (Diagnostic Ultrasound, USA). Bladder volumetrics were performed in the early morning, between 7 am and 8 am, and always prior to first ambulation after sleep. Volumetrics were performed independent of scheduled drainage tubing manipulation and Foley bag emptying, which occurs prior to 7 am on our hospital's wards. Of note, ICU nursing staff manipulate the catheter drainage tubing generally once every 1-2 hours while recording hourly urine outputs. Our hospital's ward nurse staff report manipulating the catheter bag/drainage tubing at least once per day when measuring urine output. Bladder ultrasound volumetrics were performed 5 times consecutively during each measurement session, and all values, including mean, were recorded. To validate the volumes recorded by the ultrasound device, whenever a residual urine volume greater than 10 cc was identified by ultrasound, the drainage tubing was subsequently manipulated in an up-down fashion (“milked”) to promote antegrade drainage of the residual urine for visible confirmation and measurement.

Ex-Vivo Urinary Bladder Model

To better understand the mechanism of urinary drainage through a catheter system, we designed an ex-vivo bladder model which closely mimics the relative orientation of the components of a urinary catheter/drainage tubing system in a hospital patient lying in bed. We prospectively measured the lengths, angles of curvature, and height from the floor, of each segment of the Foley catheter and drainage tubing/bag in our bed-bound hospital patients. To replicate an ex-vivo model of the catheterized bladder, a mock bladder reservoir was made using a 6L flat-bottom plastic basin. The walls were 15 cm high. The baloon end of an 18 Fr Foley catheter (BARD, USA), connected to a standard drainage tubing/bag kit (BARD, USA) was placed through a small hole in the center of the base of the basin. The edges of the hole around the catheter were sealed with silicone, such that only the patent balloon-end of the catheter resided within the interior of the basin.

Care was taken to place the catheter and bag/tubing in the same orientation as would be found in vivo. Specifically, the bag was hooked onto a laboratory bench at approximately the same height below the mock bladder as is observed clinically, such that the redundant length drainage tube possessed the same gravity-dependant “curl” just proximal from where the tube terminates into the collection bag.

To render the catheter in the same state of filling as is observed in the hospital, the bladder reservoir was filled and drained in patterns that we felt would mimic those seen in vivo. For example, to mimic initial placement of the catheter into a partially filled bladder, the basin was initially filled half-way up with water, and then allowed to drain through the drainage tube to completion. The catheter curl was then “milked” (lifted up and down). Next, to mimic the natural flow of urine from the kidney into the bladder, the basin was slowly refilled and the filling of the drainage bag and tubing and the efficacy of emptying of the basin was observed. With the drainage tube in the “curled” conformation, pressure within the proximal portion of the drainage tube was continuously measured. We performed trials with curled segment at different heights: 15 cm (10 in.), 33.5 cm (13 in.), and 37.5 cm (15 in.).

Meausurement of Flow Rate and Proximal Drainage Tube Pressure

Using the previously designed mock bladder and drainage system, we studied the drainage of the basin with the tubing either with a single “curl” or in a downward spiral conformation. Next, to measure the pressure within the proximal drainage tubing, which equals bladder pressure, we connected a small ring-shaped fitting containing a small pressure sensor electrode at the connection the Foley catheter and the proximal end of the drainage tubing. We used a tall (3 feet=100 cm) plastic column of tubing (inner diameter=3.75 cm) in order to generate sufficient antegrade pressure within the catheter and drainage tubing to overcome air-lock obstruction pressure. Initial experiments using a 15 cm tall basin as the bladder reservoir (as described in the previous section) resulted in insufficient reservoir outflow pressure to force antegrade flow across the curled drainage tube., the purpose of using a tall column of water instead of a simple 15 cm tall basin was to generate the threshold bladder outflow pressure necessary to achieve antegrade flow across the curled drainage tube.

Flow trials measuring flow rate and pressure within the proximal-most segment of the drainage tubing were performed with the drainage tubing in both the “curled” and coiled downward-spiral conformations. Pressure and flow were recorded on a continuous basis with the reservoir initially full, during emptying, and then again after “milking” the drainage tube and then refilling the empty reservoir.

Results

Bladder Ultrasound Volumetrics

Mean residual volume in hospital ward patients (M=38/F=37), were 136 ml (range 22-647 ml). A residual volume of at least 25 ml was recorded in 48% of the subjects. In the ICU patients (M=39/F=36), where urine output was measured hourly and where nursing staff routinely manipulate the Foley drainage tubing to maximize recorded urine output, mean residual volume was 96 ml (Range: 4-290 ml). A residual volume of at least 25 ml was recorded in 72%. In nearly all cases, the catheter drainage tubing was “milked” so as to induce outflow of residual urine for the purpose of confirming ultrasound findings. In this way, we confirmed the presence of a minimum of 50% of the residual volume recorded by ultrasound volumetrics.

Measurement of Flow Rate and Proximal Drainage Tube Pressure

We observed that the bottom of the curl filled with water easily, but that, immediately as the curled segment filled with urine, urine basin outflow slowed and then ceased, despite continued filling of the basin. As the basin continued to fill, the fluid meniscus at the distal limb of the curl rose, while the meniscus in the proximal limb fell. As the menisci moved in opposite directions, we hypothesized that the air pressure in the proximal limb must be increased. We tested our hypothesis by placing a needle in the gel “urine sampling port” located at the proximal end of the drainage tube (not illustrated). This caused an immediate and audible outward rush of air through the needle. Also immediately, antegrade water flow through the tube immediately resumed and the basin drained to completion.

Upon commencing filling of the reservoir at all 3 curl heights (25, 33.5, and 37.5 cm) there was an absence of antegrade flow of fluid distally into the bag. Instead, the curl would fill with fluid rapidly until the the lumen of the bottom of the curl was filled with fluid. After that, antegrade flow from the reservoir decreased, while simultaneously, the meniscus in the distal segment of the curl rose. Ultimately, distal antegrade flow commenced when a peak pressure was reached in each trial. The peak pressures, defined as the maximum pressure within the proximal-most segment of the drainage tube, corresponding to the pressure required to overcome the air-lock and re-establish distal antegrade flow of fluid into the collection bag, was recorded as 23.20 cm H₂O) at curl height of 25 cm, 30.63 cm H₂O for curl height 33.5 cm, and 34.16 cm H₂O at curl height of 37.5 cm.

With the drainage tube in the downward-spiral coiled conformation, there was a continuous and equal antegrade flow both proximally and distally throughout, reflecting the absence of any temporary complete obstruction to outflow.

Discussion

Urethral catheters are used for the purpose of maintaining the bladder empty of urine, at all times. They should allow unobstructed and complete urine outflow during conditions of ambulation and bedrest. Our findings show how, in most circumstances, current catheter/drainage tubing systems do not drain the bladder completely or efficiently.

A catheter tubing system is typically comprised of a urethral Foley catheter, and a drainage tubing unit which terminates in a collection bag. It is desirable that this system provide: 1) Low pressure bladder drainage; 2) Continuous and complete bladder drainage, and 3) The system be “closed” from the environment, in order to minimize the risk of infection to the patient.

To accomplish these objectives the catheter drainage tubing is typically vented, e.g., to the atmosphere, so that air within the tubing can exit distally through the vented bag as urine fills the tubing; In addition, it is desirabel that the entire length of drainage tubing be free of air-locks.

Current catheter drainage tubing units do not continuously drain the bladder due to the presence of air-locks within the tubing system, and large undrained urine residual volumes typically develop.

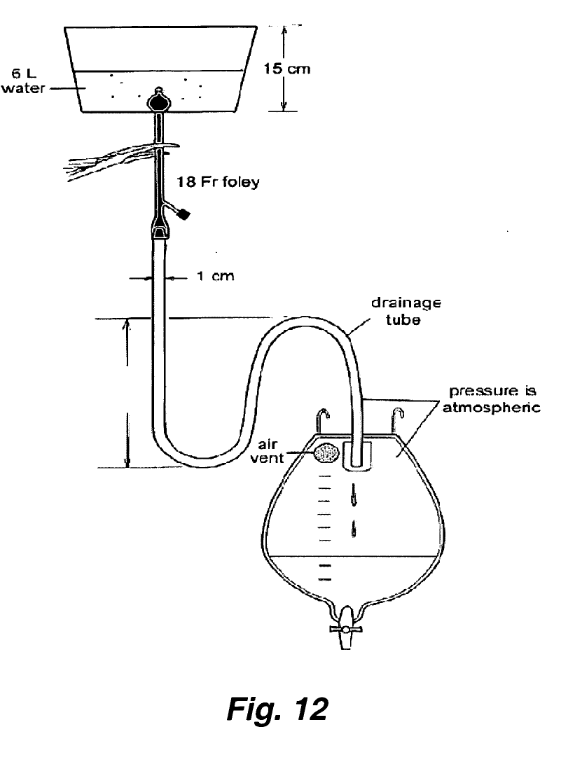

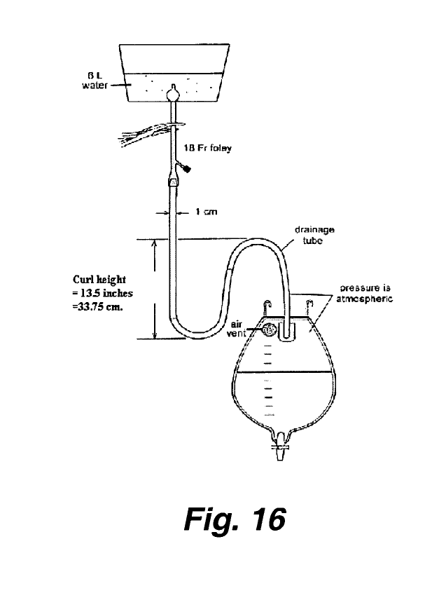

The collection bags of the leading US manufacturers' drainage tubing units are vented (see FIGS. 12-16), so that the intraluminal pressure of the bag and empty drainage tubing equals atmospheric pressure. When urine enters the catheter and drainage tubing from the bladder, it displaces an equal volume of intralunimal air. If this displaced has no way to exit the system, it will remain in the system, but under higher pressure. Over time, as more urine enters the drainage system from the bladder, the pressure of the trapped air steadily increases (FIG. 16).

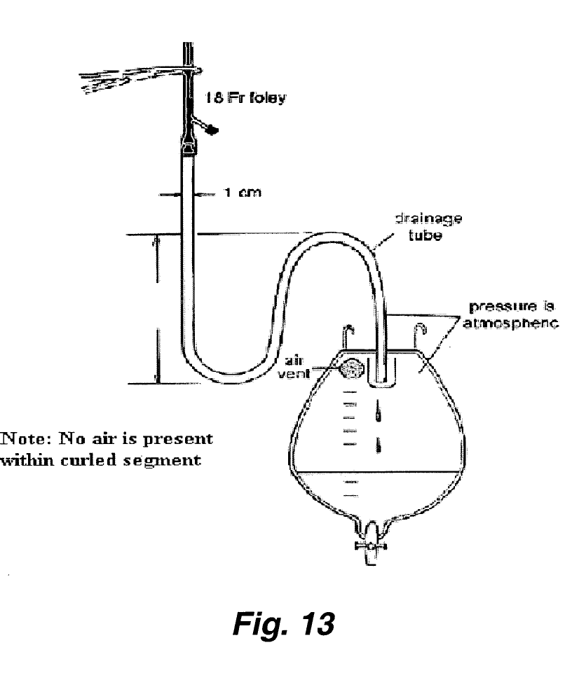

If the tubing system is vented, this displaced air can exit distally, through the collection bag, without an increase in intraluminal pressure. For example, when a Foley catheter is placed into a patient with a full bladder, the drainage tubing is full of air, which is in direct continuity to the vented bag. Therefore, air within the entire tubing system is atmospheric (see FIG. 12). Upon entering the bladder, the Foley catheter and drainage tubing immeditely fill with urine. The air within the tubing system prior to catheter placement is all displaced distally, and exits through the vented bag. Note the absence of air within the and the drainage tubing as the bladder empties (see FIG. 13).

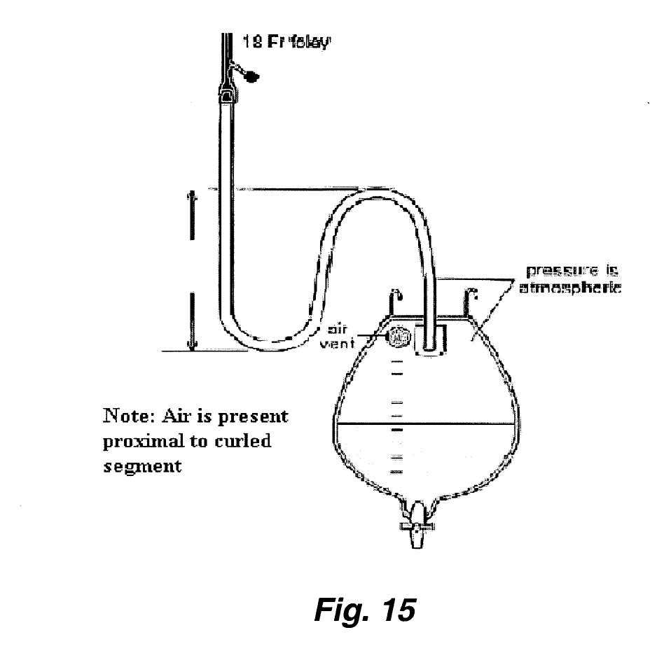

Air-locks serve to separate intraluminal air into pockets, such that air proximal to a dependent curl is completely “cut-off” from air within the distal segment, which opens into the vented collection bag (FIGS. 15-16). As illustrated in FIGS. 15 and 16, as new urine enters the tubing system, it must displace intraluminal air within the proximal segment of the drainage tubing.

If an air-fluid level is present at the bottom of a dependent curl, there is no way for the ddisplaced air to exit distally. Instead, as progressively more urine attempts to enter the proximal segment of the drainage tubing, the air pressure within the proximal tubing segment merely increases. For the air pocket to be displaced distally, a force must be available to “push” the fluid column distal to the air-pocket distally, against gravity (see FIG. 16). Naturally, the greater the height distance the distal fluid column must travel, the more pressure will need to be exerted from the proximal end to establish antegrade flow.

The air proximal to the air-fluid level is thus trapped, and increases in pressure as more and more incoming urine forces the intraluminal air to occupy a progressively smaller volume. Intraluminal pressure increases as the bladder fills with urine. This is evidenced by assymetric menisci levels across the air-fluid level (see FIG. 16). Air within the drainage tubing proximal to the air-lock can only be compressed so much, so that very quickly, the limiting force to antegrade drainage is bladder pressure. The bladder is naturally compliant, and therefore it responds to higher distal pressures by stretching and accomodating greater (undrained) urine volumes while maintaining a low intravesical pressure. Eventually, the bladder's compliance threshold is met, so that additional urine inflow from the ureters is subjected to progressively higher intravesical pressures. As bladder pressure increases, the comumn of fluid distal to the air-fluid level is pushed upward, against gravity, to establish antegrade flow. Eventually, the proximal meniscus is pushed upward to the apex of the curl. When the distal meniscus reaches the apex, it drains fluid across the apex, and down the distal curl toward the bag. Further pressure from the bladder sustains the distal meniscus at this height, and perpetuates this “overflow” pattern of drainage. Note that the bladder pressure must remain high throughout, to sustain overflow drainage. The high bladder pressure is a consequence of a full state at maximal compliance bladder wall compliance.

Three key elements contribute to the formation of airlocks:

1. The drainage tubing is positioned in such a way that there is a dependent U-shaped “curled” segment.

2. The distal-most fluid meniscus is below the highest point of the curled of U-shaped tubing conformation (i.e. the meniscus lies below any tubing distal to it).

3. When newly produced urine drains into the drainage tubing and pools at the dependent segment, filling the lowest curled segment, results in two separate menisci.

First, let us examine the sequence of events within the catheter system immediately preceeding and following insertion of the catheter into the patient's bladder.

FIG. 12 illustrates the semi-full mock bladder reservoir immediately after the catheter was opened to drainage, thus simulating insertion and prompt drainage of the bladder. The catheter drainage tubing is full of fluid throughout, and the rate of drainage out of the reservoir into the tubing equals the rate of drainage of fluid out of the tubing into the collection bag. Note the absence of air within the drainage tubing system.

FIG. 13 shows the fluid columns when the bladder is finally empty (simulated experimentally by applying a clamp to the distal catheter): the rate of fluid draining from the bladder catheter into the proximal segment of drainage tubing slows to zero, leaving a standing column of fluid within the proximal segment of tubing. The antegrade movement of fluid within the distalmost segment of tubing is now also slowed to a standstill. However, because this segment is oriented vertically, the fluid column within the tubing segment responds to gravity and “falls” directly into the bag, emptying the distal tubing segment from bottom (bag level) to the apex, where a meniscus forms at the top of the vertical segment. Drainage of the fluid within the tubing system is in perfect balance, such that every drop of newly produced urine that enters the bladder from the ureters will drain into the catheter and drainage tube and cause overflow “waterfall” drainage of an equal volume across the distally located meniscus of the vertical tube segment (FIG. 13). In essence, for fluid to drain across the waterfall meniscus, it does not need to travel upward against gravity, since the fluid level is flatly spread across and up to the edge of the highest point of the tubing. The balance of inflow equaling outflow established across the distally located “waterfall ” meniscus will continue to offer complete bladder drainage, as long as the distal meniscus of the system is at the apex of the curled tubing, as illustrated in FIG. 13.

This balance of tube inflow equaling outflow is lost the moment that the tubing is moved (such as with ambulation, or by the hand of the physician or nurse when the bag and tubing are manipulated during routine emptying of the bag for measurement of voided volume. For example, nurses routinely “milk” the tubing while emptying the bag, in an effort to drain all visible urine within the tubing into the bag so as to maximize the recorded urine output. “Milking” the tubing refers to lifting the curled segment of tubing, which invariable contains some amount of pooled urine, upward and downward, to allow the pooled urine to drain into the bag. The tube is moved in this way until urine is no longer visible within the drainage tubing. The vast majority of nurses at our institution report this practice on a routine basis, and, nearly all report that, routinely, such “milking” of the catheter results in drainage of additional large volumes or urine, such that the “milked” urine output from the tubing system accounts for an average of 30-50% of the total recorded output, and has frequently resulted in sudden drainage of volumes in excess of 200-1000 milliliters.

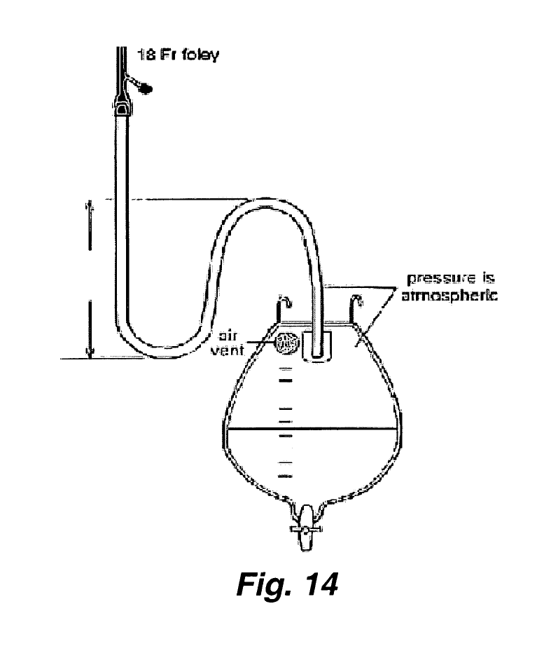

In the absence of an obvious source of mechanical obstruction, such as particulate matter or compression occlusion of the drainage tubing, the presence of undrained urine within the bladder is a result of an air-lock within the tubing system. Specifically, the air-lock forms whenever the three criteria listed above are met. FIG. 14 illustrates the manually empted drainage tubing system, curled in a U-shape.

FIG. 15 illustrates the system as newly produced urine from the bladder has trickled down the drainage tube, and pooled at the dependent-most segment of the curled tubing. The moment that the cross-section of the dependent-most segment of tubing is filled with fluid, two separate menisci are formed. Furthermore, and perhaps most importantly, the air within the segment of tubing proximal to (i.e. closer to the bladder) is the dependent segment is no longer in continuity with the air in the segment distal to the dependent segment. However, note that in FIG. 5 the height of the menisci is asymetric heights: the proximal meniscus (on left) is lower than the distal meniscus (on right). This is due to the effect of the air-lock. As urine from the bladder drains into the catheter, to enter the proximal segment of tubing it must displace an equal volume of the air presently residing within the proximal segment. Due to the presence of pooled fluid at the dependent curl, this displaced air cannot escape distally along the tubing and into the vented bag. This trapped volume of air from the proximal tubing segment serves as the air-lock described. Hence, the air within the proximal segment cannot escape to accommodate the passage of fluid, and as progressively more fluid collects within the bladder, awaiting antegrade drainage, the pressure of the air within the proximal segment increases. Due to the high compliance of the bladder wall, large volumes of undrained fluid can be stored within the bladder at low pressures, in response to the air-lock obstruction. Ultimately, however, the stretching effect of the increasing bladder volume either induces a spasmic contraction, or, it approaches its own maximum stretched volume, and ruptures.

Fortunately, an awake patient is usually able to either feel when their bladder volume approaches maximum capacity, or they ambulate or otherwise disturb the curled conformation of the tubing so that, at least temporarily, the air-lock can be overcome and limited drainage can be effected. In unconscious patients, the frequency of nursing checks to empty the collection bag and calculate in's/outs is sufficient to prevent obstruction to the point of bladder rupture. However, based on the findings from ultrasound evaluation of catheterized patients hospitalized at our institution, all bedbound patients had substantial undrained urine residual volumes.

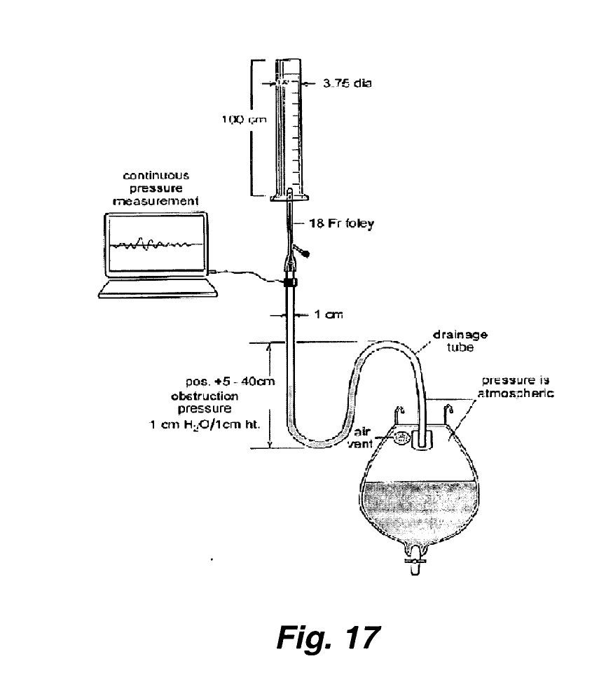

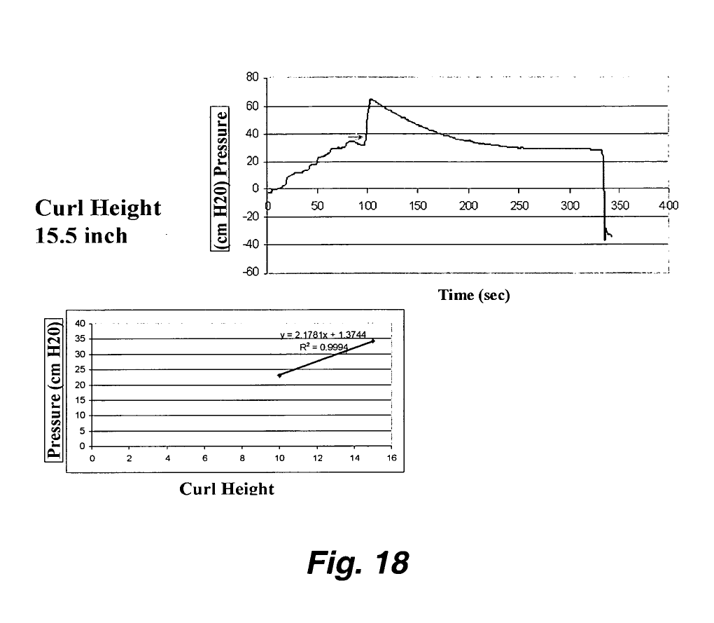

We sought to quantify the magnitude of the obstruction caused by the air-lock. To do so, we connected a pressure transducer in-line between the catheter and the drainage tubing (FIG. 16). The catheter was connected to a mock urinary bladder, as described. The mock bladder was open to the environment. The pressure transducer was connected to a computer/software unit that calculated pressure continuously (FIG. 17). After an airlock was created, with the height of the curled segment measuring 15.5 inches (38.75 cm), water was slowly dribbled into the mock bladder as pressure within the proximal segment of the drainage tubing was recorded continuously during filling (FIG. 7). No antegrade flow from the tubing into the drainage bag occurred below the pressure indicated by the red arrow. The arrow in FIG. 18 indicates the pressure at which the air-lock was overcome and antegrade flow commenced. The pressure spike beyond the arrow is artifact (from filling the reservoir). It is important to note that the pressure within the proximal segment remains fixed at an elevated baseline pressure (˜36 cm H2O) throughout, after the air-lock is overcome. This is consistent with our clinical ultrasound findings where in many patients, despite regular urine outflow drainage from the catheter, the bladder often contained a large residual-volume. We conducted many such pressure-flow trials, varying the height of the curled segment. We found that a linear relationship existed between the height of the curled segment, and the pressure required to overcome the air-lock. Roughly, for every centimeter of curl height, an obstruction pressure of 1 cm water must be overcome to overcome the air-lock. For the curl height of 38.75 cm illustrated in FIG. 8, an air-lock obstruction pressure of ˜36 cm H2O was measured.

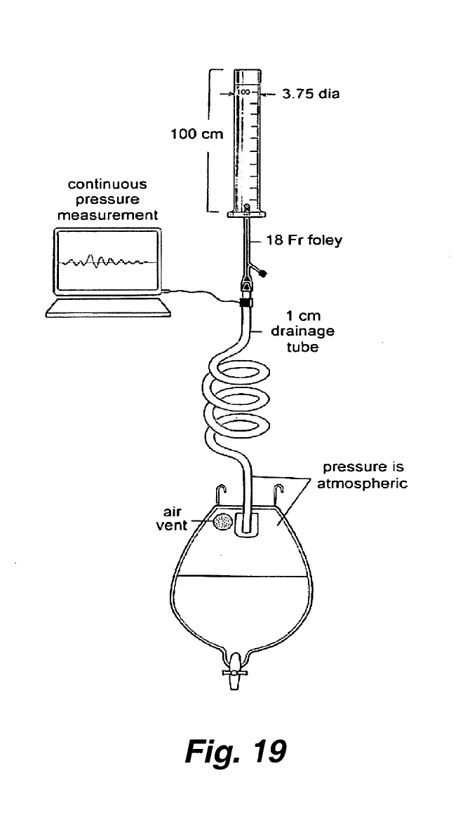

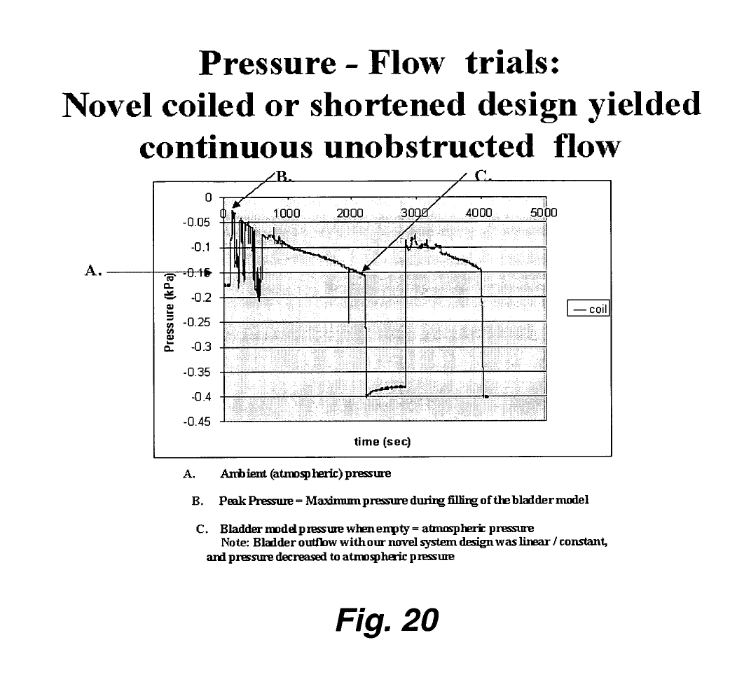

Once we identified air-locks as the occult source of obstructed urine outflow in catheterized patients, and we understood how they developed as a result of a curled, dependent segment of drainage tubing, we proceeded to design a drainage tube system that would provide antegrade drainage of fluid without the curled, dependent segments that lead to air-locks. A simple solution is to make the drainage tube in a coiled downward spiral shape, so that all points along the drainage tube allow for downward gravity dependant flow (FIG. 19). The absence of any dependent curled segments assures that flow along the tubing segment will continue downward into the collection bag, uninterrupted. We measured the pressure within the proximal segment during emptying of the ex-vivo mock urinary b ladder used previously, and we demonstrated that flow is continuous and complete at all times when our proposed coiled drainage tubing is used (FIG. 20). Note that pressure within the drainage tube is constantly decreasing during emotying, and reaches zero once the mock urinary bladder is empty.

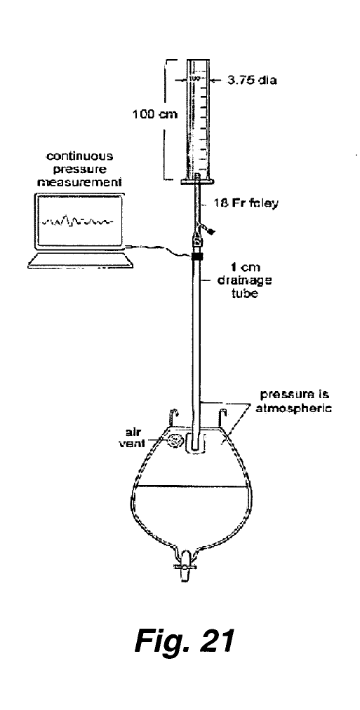

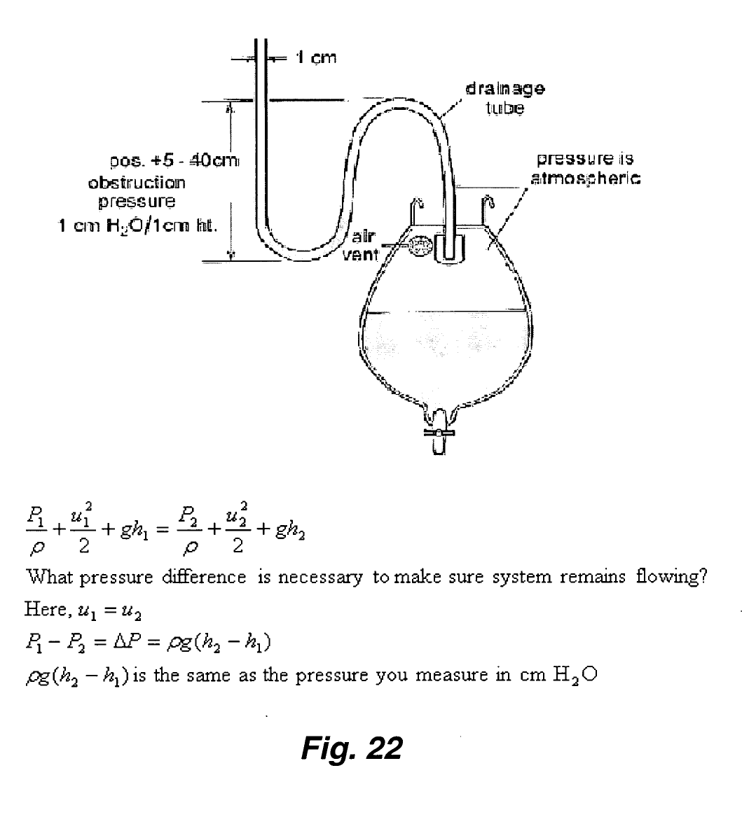

An alternative design is to simply shorten the drianage tube. After all, just as with the coiled conformation, the shortened drainage tube does not allow for dependent curls. The benefit of the coiled conformation is that the coils allow the tubing to be stretched as desired when additional length is needed, while maintaining a downward gravity-dependent trajectory throughout (FIG. 21).

The incidence and magnitude of the residual urine volumes observed in our study have important implications for hospitalized, marginally ambulatory, and/or bedbound patients, and likely contributes to the problems associated with acute and chronic Foley catheter use.

The risk of UTI increases with increasing duration of catheterization. Recurrent infections lead to bacterial resistance to antibiotics. Long term catheterization has been associated with severe complications such as pyelonephritis, nephrolithiasis, epididymitis and prostatitis. Bacteremia can occur when large, static urine volumes become infected, and such urine is exposed to sites of chronic local urothelial trauma. Factors such as catheter erosion, focal bladder wall ischemia caused by persistent increased intraluminal pressures, and acute trauma from excessive catheter traction, as serve as mechanisms for the development of sepsis.

A urethral catheter that simply fails to adequately drain the bladder intermittently can easily lead to significant morbidity and mortality. Airlock obstruction is especially insidious because it often goes un-noticed as a cause for discomfort when the patient's urine in unremarkable in appearance to suggest an obvious cause for obstruction (obstructing clots, heavy sediment, etc.). For example, intermittent airlock obstruction of a catheter leads to acute episodes of painful bladder distension, which can cause the unsupervised obtunded/disoriented/or senile patient to respond by traumatically removing his/her catheter with the balloon intact. The resulting urethral trauma, bleeding are risk factors for bacteremia, and nearly always lead to emergency medical/urologic consultation. Such patients are also at risk for developing a urethral stricture, a sequellae which alone can account for significant chronic morbidity, poor quality of life, and the expenditure of vast healthcare resources.

Despite increasing numbers of patients with chronic indwelling Foley catheters, product innovation to address this problem has generally been limited to bacteriostatic and bacteriocidal coatings, designed to impede bacterial migration along the catheter into the patient. Such coatings have generally been applied to the urethral catheters. However, the efficacy of these products to prevent UTI's during short-term catheter use has been inconclusive. Coated chronic indwelling catheters have failed to demonstrate efficacy to prevent UTI's, and this, combined with their significantly higher cost, has limited their use overall. Manufacturers of the one-piece drainage tubing and bag lits have also coated the drainage tubing with bacteriostatic and/or bactericidal agents. Such coated tubing products have not demonstrated the ability prevent UTI's.

All such coated catheters and drainage tubing units are limited by the fact that they are limited to attempting to stop the relatively inevitable ascension of bacteria along the catheter into the patient. The externalized catheter surface will always become contaminated with bacteria, and a proportion of these will frequently ascend to the level of the lower urinary tract. Thus, the critical issue becomes minimizing the liklihood that such ascending bacteria will lead to urinary tract infection infection. Indeed, once even a few bacteria ascend into the patient's bladder, the moment bladder residual urine becomes infected, the catheter's bacteriostatic or bacterioccidal coating becomes useless.

Blockage is problem frequently reported by more than half of outpatients with chronic urinary catheters. The literature suggets that the most common causes of catheter blockage include blood clots, sediment crystals and mucus within the catheter lumen. Air-locks could be the unrecognized primary cause of obstruction, while observed clots, mucus and sediment develop secondary to stasis caused by air-lock obstruction.

Catheter blockage accounts for many unscheduled office, evening and weekend visits, in addition to emergency room visits and visits by home nurses. A study examining after-hours home care nursing calls notes that 22 of 25 patients reported catheter-related problems.

Catheter urine outflow obstruction is harmful to the patient at many different and synergistic levels. In addition to predisposing the patient to infections, high intravesical pressures and large residual volumes directly and indirectly damage the upper and lower urinary tracts, alter local anatomy and diagnostic evaluations, and can lead to long term sequellae such as local scarring and stricture.

Within the bladder, the high intravesicle pressures that occur with bladder distension can defunctionalize bladder smooth muscle and cause acute urinary retention and chronic bladder dysfunction. Unless scheduled catheter removal for such patients occurs early during the day, subsequent urinary retention (which may develop several hours later) will necessitate after-hours or medical attention, adding to health-care costs. Acute bladder obstruction can also cause autonomic dysreflexia, committing the patient to a host of interventions and medical costs. Acute obstruction can also cause unstable blader contractions, potentially prompting a confused patient to pull his/her catheter. Resulting trauma can lead to unecessary diagnostic studies and associated costs, prolonged hospital stay, urethral strictures and/or bacteremia. An obstructed bladder can damage the upper tracts by limiting drainage, causing nephropathy. Resulting urinary stasis promotes nephrolithiasis, etc. If an indwelling ureteral stent is in place, then high intravesical pressure is transmitted through the patent stent to the upper tracts (FIG. 12), potentially causing nephropathy, hydronephrosis and scarring, urine stasis, etc. Our experiments show that the greater height of the curled segment, the greater the positive (obstructing) pressure within the proximal segment will be.

Unrecognized urine outflow obstruction from the upper and lower tracts can influence a patient's physical examination findings, and potentially lead to misdiagnosis. Intraoperatively, an unexpectedly distended bladder can lead to iatrogenic injury or misdiagnosis if the surgeon assumes that it is decompressed based on the fact that the catheter is in place. Catheter/drainage tube obstruction can also cause falsely low urine output, which will alter recorded fluid totals and mislead fluid management both intraoperatively and postoperatively. Suboptimal catheter drainage can create the symptoms and findings that lead mislead management toward the unncessary administration of diuretics, fluids, analgesics, radiographic studies, etc. Such interventions can cause secondary adverse medical outcomes, such as the exacerbation of CHF, electrolyte imbalances, etc.

Another unexpected potential complication that can result from the high intravesical pressures caused by an obstructed catheter drainage system is leakage across “fresh” surgical wounds and anastomoses within the urinary tract. For example, if extravasation of urine at the vesico-urethral anastomosis after prostatectomy did occur, could this contribute to the development of bladder-neck contractures, given the highly sclerotic effects of urine on surrounding tissues? The inflammatory response to urine extravasated into the prostatic bed could also harm the exposed neurovascular bundles. Leakage across ureteral anastomoses would be expected to contribute to subsequent stricture formation. In sum, high bladder pressure due to catheter outflow obstruction can promote and prolong leakage from any surgical site within the urinary tract.

Studies of catheter drainage characteristics in pediatric and neonatal patient populations are currently underway at our institution. The narrower and often curled catheters frequently used with these patients suggests that the drainage tubing outflow may also be compromised.

Conclusions

Traditional Foley catheter drainage does not consistently evacuate the urinary bladder. Increased residual urine likely contributes to nosocomial urinary tract infections, and many other associated problems with Foley catheter drainage. Our novel Foley drainage tubing system eliminates obstructive air-locks and help to optimally drain the urinary bladder.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A system for improved drainage from a bladder in a patient, said system comprising:
a fluid collection apparatus;
a drainage receptacle; and
a connecting tube comprising a means for reducing or eliminating airlocks in said connecting tube and thereby providing sufficiently low backpressure such that a patient having a urinary bladder drained with said system maintains an average residual bladder urine volume of less than about 50 cubic centimeters over a period of at least four hours after initial drainage without manipulation of components of said system.

2. The system of claim 1, wherein said means provides sufficiently low backpressure such that a patient having a urinary bladder drained with said system maintains an average residual bladder urine volume of less than about 25 cubic centimeters over a period of at least eight hours after initial drainage without manipulation of components of said system.

3. The system of claim 1, wherein said collection apparatus comprises a Foley catheter.

4. The system of claim 1, wherein said drainage receptacle comprises a urine collection bag.

5. The system of claim 1, wherein said drainage receptacle comprises a vented urine collection bag.

6. The system of claim 1, wherein said means for reducing or eliminating airlocks comprises a means for producing a downward spiral shape in said connecting tube.

7. The system of claim 6, wherein said means for reducing or eliminating airlocks comprises an external semi-rigid coil through which said connecting tube is threaded.

8. The system of claim 6, wherein said means for reducing or eliminating airlocks comprises an external semi-rigid coil to which said connecting tube is attached.

9. The system of claim 6, wherein said means for reducing or eliminating airlocks comprises a semi-rigid coil formed from all or a part of said connecting tube.

10. The system of claim 1, wherein said means for reducing or eliminating airlocks comprises a tensioner attached to said connecting tube.

11. The system of claim 10, wherein said tensioner comprises a spring or elastic strap attached to said connecting tube.

12. The system of claim 11, wherein said tensioner is removably attached to said connecting tube.

13. The system of claim 11, wherein said tensioner comprises a clip for attachment to bedding, a bedside, or an iv stand.

14. The system of claim 1, wherein said means for reducing or eliminating airlocks comprises an elastic or elasticized bellows tubing.

15. The system of claim 1, wherein said means for reducing or eliminating airlocks comprises a form for wrapping excess collection tubing.

16. The system of claim 15, wherein said form holds excess collection tubing in a downward spiral shape.

17. The system of claim 1, wherein said means for reducing or eliminating airlocks comprises a tubing auto-winder.

18. A medical drainage device or system comprising a connecting tube, said connecting tube comprising a means for reducing or eliminating airlocks in said connecting tube and thereby providing sufficiently low backpressure such that when a patient is equipped with said device or system to drain a bladder, said patient maintains an average residual bladder urine volume of less than about 50 cubic centimeters over a period of at least four hours after initial drainage without manipulation of components of said system.

19. The drainage device or system of claim 18, wherein said means for reducing or eliminating airlocks comprises a means for producing a downward spiral shape in said connecting tube.

20. The drainage device or system of claim 19, wherein said means for reducing or eliminating airlocks comprises an external semi-rigid coil through which said connecting tube is threaded.

21. The drainage device or system of claim 19, wherein said means for reducing or eliminating airlocks comprises an external semi-rigid coil to which said connecting tube is attached.

22. The drainage device or system of claim 19, wherein said means for reducing or eliminating airlocks comprises a semi-rigid coil formed from all or a part of said connecting tube.

23. The drainage device or system of claim 18, wherein said means for reducing or eliminating airlocks comprises a tensioner attached to said connecting tube.

24. The drainage device or system of claim 23, wherein said tensioner comprises a spring or elastic strap attached to said connecting tube.

25. The drainage device or system of claim 24, wherein said tensioner is removably attached to said connecting tube.

26. The drainage device or system of claim 24, wherein said tensioner comprises a clip for attachment to bedding, a bedside, or an iv stand.

27. The drainage device or system of claim 18, wherein said means for reducing or eliminating airlocks comprises an elastic or elasticized bellows tubing.

28. The drainage device or system of claim 18, wherein said means for reducing or eliminating airlocks comprises a form for wrapping excess collection tubing.

29. The drainage device or system of claim 28, wherein said form holds excess collection tubing in a downward spiral shape.

30. The drainage device or system of claim 19, wherein said device or system further comprises a drainage device selected from the group consisting of a urinary catheter, a chest tube, a mediastinal tube, and a nasogastric tube.

31. The drainage device or system of claim 19, wherein said device or system further comprises a urinary leg bag.

32. The drainage device or system of claim 31, wherein said device or system further comprises a urinary leg bag, said leg bag comprising a venting tube.

33. A urinary catheter comprising a connecting tube, wherein said connecting tube comprises a downward spiral shape.

34. A urinary leg bag comprising a connecting tube and a venting system, wherein said connecting tube comprises a downward spiral shape and said venting system comprises a venting tube.

35. A collecting tube for use in a medical drainage device, wherein said collecting tube comprises a means for reducing or eliminating airlocks in said tube.

36. The collecting tube of claim 35, wherein said means for reducing airlocks comprises a means of producing a downward spiral shape in said collecting tube.

37. The collecting tube of claim 36, wherein said tube comprises means for attachment to a catheter.

38. The collecting tube of claim 36, wherein said tube comprises means for attachment to a Foley catheter.

39. The collecting tube of claim 36, wherein said tube comprises means for attachment to a waste receptacle for biological fluids.

40. The collecting tube of claim 36, wherein said means for a means for producing a downward spiral shape comprises an external semi-rigid coil through which said connecting tube is threaded.

41. The collecting tube of claim 36, wherein said means for a means for producing a downward spiral shape comprises an external semi-rigid coil to which said connecting tube is attached.

42. The collecting tube of claim 36, wherein said means for a means for producing a downward spiral shape comprises a semi-rigid coil formed from all or a part of said connecting tube.

43. The collecting tube of claim 35, wherein said means for reducing airlocks comprises a tensioner attached to said connecting tube.

44. The collecting tube of claim 43, wherein said tensioner comprises a spring or elastic strap attached to said connecting tube.

45. The collecting tube of claim 44, wherein said tensioner is removably attached to said connecting tube.

46. The collecting tube of claim 44, wherein said tensioner comprises a clip for attachment to bedding, a bedside, or an iv stand.

47. The collecting tube of claim 35, wherein said means for reducing or eliminating airlocks comprises an elastic or elasticized bellows tubing.

48. The collecting tube of claim 35, wherein said means for reducing or eliminating airlocks comprises a form for wrapping excess collection tubing.

49. The collecting tube of claim 48, wherein said form holds excess collection tubing in a downward spiral shape.

50. A method of reducing urinary tract infection in a subject bearing a urinary catheter, said method comprising:
providing a connecting tube coupled to said catheter where said a connecting tube comprises a means for reducing or eliminating airlocks in said connecting tube and thereby providing sufficiently low backpressure such that a patient having a urinary bladder drained with said system maintains an average residual bladder urine volume of less than about 50 cubic centimeters over a period of at least four hours after initial drainage without manipulation of components of said system.

51. The method of claim 50, wherein said means provides sufficiently low backpressure such that a patient having a urinary bladder drained with said system maintains an average residual bladder urine volume of less than about 25 cubic centimeters over a period of at least eight hours after initial drainage without manipulation of components of said system.

52. The method of claim 50, wherein said catheter is a Foley catheter.

53. The method of claim 50, wherein said means for reducing or eliminating airlocks comprises a means for producing a downward spiral shape in said connecting tube.

54. The method of claim 53, wherein said means for reducing or eliminating airlocks comprises an external semi-rigid coil through which said connecting tube is threaded.

55. The method of claim 53, wherein said means for reducing or eliminating airlocks comprises an external semi-rigid coil to which said connecting tube is attached.

56. The method of claim 53, wherein said means for reducing or eliminating airlocks comprises a semi-rigid coil formed from all or a part of said connecting tube.

57. The method of claim 50, wherein said means for reducing or eliminating airlocks comprises a tensioner attached to said connecting tube.

58. The method of claim 57, wherein said tensioner comprises a spring or elastic strap attached to said connecting tube.

59. The method of claim 58, wherein said tensioner is removably attached to said connecting tube.

60. The method of claim 58, wherein said tensioner comprises a clip for attachment to bedding, a bedside, or an iv stand.

61. The method of claim 50, wherein said means for reducing or eliminating airlocks comprises an elastic or elasticized bellows tubing.

62. The method of claim 50, wherein said means for reducing or eliminating airlocks comprises a form for wrapping excess collection tubing.

63. The method of claim 62, wherein said form holds excess collection tubing in a downward spiral shape.

64. The method of claim 50, wherein said means for reducing or eliminating airlocks comprises a tubing auto-winder.

65. A kit for draining a biological fluid from a site in a subject, said kit comprising:
a collecting means for application to said site; and
a connecting tube comprising a means for reducing or eliminating airlocks in said connecting tube and thereby providing sufficiently low backpressure such that a patient having a urinary bladder drained with said connecting tube maintains an average residual bladder urine volume of less than about 50 cubic centimeters over a period of at least four hours after initial drainage without manipulation of components of said system.

66. The kit of claim 65, further comprising a waste receptacle for receiving biological fluid drained from said site.

67. The kit of claim 65, wherein said collecting means is a Foley catheter.

68. The kit of claim 65, wherein said collecting means is selected from the group consisting of a Foley catheter, a Jackson Pratt tube, and a nasogastric tube.

69. The kit of claim 65, wherein said kit further comprises instructional materials teaching the use of said drainage device with said collecting tube.

70. An iv stand, said iv stand comprising a container or support for containing or holding a waste receptacle for biological fluids.

71. The iv stand of claim 70, wherein said iv stand is on wheels.
